(19) United States
(12) Patent Application Publication
(10) Pub. No.: US 2026/0115442 A1
(43) Pub. Date: Apr. 30, 2026

(54) INFILTRATING MEDICINAL LIQUID SUPPLY ULTRASONIC CAVITATION THERAPEUTIC PATCH DEVICE

(71) Applicant: Zhijun Peng, Foshan (CN)

(72) Inventor: Zhijun Peng, Foshan (CN)

(21) Appl. No.: 18/983,387

(22) Filed: Dec. 17, 2024

(30) Foreign Application Priority Data
Oct. 30, 2024 (CN) 202411529919.X

(51) Int. Cl. A61M 37/00

(52) U.S. Cl. CPC A61M 37/0092; A61M 2037/0007

(57) ABSTRACT

An infiltrating medicinal liquid supply ultrasonic cavitation therapeutic patch device comprises a patch, an infiltrating medicinal liquid cotton pad, an ultrasonic therapeutic unit, and a medicinal application box. The medicinal application box includes a box body and a box cover, with the box body further featuring a medicinal chamber and medicinal liquid outlet holes. The box cover is designed to open and close over the medicinal chamber, and the patch includes through-holes. By employing a built-in liquid supply mechanism, the device eliminates the constraints of external electric liquid supply mechanisms and conduits, achieving an integrated design of medicinal application and ultrasonic therapy.

### FIELD OF THE INVENTION

The present invention relates to the field of massage and physiotherapy devices, specifically to a therapeutic instrument integrating medicinal application and ultrasonic cavitation therapy.

### BACKGROUND OF THE INVENTION

With the continuous development of human society, the pace of life has accelerated significantly. Prolonged overtime work, sleep deprivation, and maintaining static postures for extended periods often cause significant blood circulation issues within the human body, leading to varying degrees of soreness, pain, gout, and other discomforts in certain body parts. Over time, these conditions may result in suboptimal health, fatigue, and other sub-health symptoms. In daily life and work, individuals may also experience injuries such as swelling or bruising due to accidental falls, collisions, or other incidents. These symptoms are often alleviated using traditional medicinal liquids applied externally, but the therapeutic effect is typically slow.

On Apr. 7, 2020, the applicant filed a patent application with the China National Intellectual Property Administration (Application No. 202010265165.7, Publication No. CN116672239A) entitled “FLEXIBLE AND ADJUSTABLE AIRBAG ULTRASONIC MASSAGE HEALTHCARE DEVICE.” This therapeutic device comprises a wearable unit and an ultrasonic massage head mounted on the wearable unit. The ultrasonic massage head includes an inner cavity and is equipped with components such as a quick-connect air tube fitting and multiple jet nozzles. An ultrasonic motor or transducer is installed within the massage head. During use, the device allows simultaneous medicinal liquid injection and high-frequency ultrasonic vibration massage therapy on targeted body areas. The high-frequency vibrations facilitate the opening of skin pores, enabling faster absorption of the injected medicinal liquid. Additionally, the vibrations promote blood circulation and metabolism, enhancing therapeutic efficiency and accelerating the relief of pain, swelling, and other symptoms.

However, the above-mentioned therapeutic device has several shortcomings, outlined as follows:

First, an additional electric medicinal liquid supply mechanism is required to provide liquid to the ultrasonic massage head. The connection between the ultrasonic massage head and the supply mechanism requires pipelines, which, along with the associated wires and components, restricts portability and convenience. Consequently, users cannot perform therapy while engaging in other activities, such as working.

Second, excessively long pipelines in the system often lead to adhesion and loss of medicinal liquid on the inner walls of the pipelines. This results in wastage of expensive medicinal liquids, increasing costs without achieving the intended therapeutic effect.

Third, the direct contact between the ultrasonic motor or transducer and the medicinal liquid within the ultrasonic massage head not only increases liquid consumption but also shortens the lifespan of the motor or transducer. Prolonged adhesion of the medicinal liquid to these components further complicates maintaining cleanliness and hygiene.

In light of these deficiencies, the applicant believes it is necessary to improve and redevelop this type of therapeutic device to better meet the health needs of users.

Here's the English translation of the provided section, tailored for a USPTO patent application:

### SUMMARY OF THE INVENTION

The objective of the present invention is to address the aforementioned problems and deficiencies by providing an infiltrating medicinal liquid supply ultrasonic cavitation therapeutic patch device. This therapeutic patch integrates medicinal application and ultrasonic therapy through a built-in liquid supply mechanism. It enables simultaneous medicinal application and ultrasonic cavitation therapy on human body parts, offering the advantages of easy wearability, convenience, and compatibility with daily activities such as household chores and work. The design also facilitates simple replacement of the infiltrating medicinal liquid cotton pad, allowing repeated use of the device. Moreover, the independent design of the medicinal liquid chamber and the ultrasonic therapeutic unit ensures enhanced lifespan and hygiene of the device.

The technical solution of the invention is implemented as follows: An infiltrating medicinal liquid supply ultrasonic cavitation therapeutic patch device, characterized by comprising a patch and a medicinal application box mounted on the patch, the box being equipped with an infiltrating medicinal liquid cotton pad and an ultrasonic therapeutic unit. The medicinal application box includes a box body and a cover that are engaged with each other. The box body is further provided with a medicinal chamber for housing the infiltrating medicinal liquid cotton pad. The bottom surface of the box body is equipped with several medicinal liquid outlet holes connecting to the medicinal chamber. The box cover is designed to open and close over the medicinal chamber, forming a built-in liquid supply mechanism with the cover, the infiltrating medicinal liquid cotton pad, the medicinal chamber, and the outlet holes. The ultrasonic therapeutic unit is mounted on the box body.

Further, the bottom surface of the box body is equipped with a cavity chamber for embedding the ultrasonic therapeutic unit, which can be detachably installed within the cavity chamber.

Additionally, snap-fit structures, comprising locking protrusions and slots, are provided between the ultrasonic therapeutic unit and the cavity chamber.

Furthermore, the box body features a through-hole connecting the cavity chamber, with a limiting step formed between the through-hole and the cavity chamber.

The advantageous effects of the invention are as follows: the invention achieves an integrated design of medicinal application and ultrasonic therapy by employing a built-in liquid supply mechanism. This eliminates the need for traditional external liquid supply systems with electric pumps and conduits, enabling simultaneous medicinal application and ultrasonic cavitation therapy for various parts of the body without being restricted by wires or pipelines. It is highly portable, allowing users to wear it comfortably and engage in daily activities such as household chores and work. Additionally, the simple replacement of the infiltrating medicinal liquid cotton pad accommodates the use of different therapeutic liquids or replenishment with new medicinal liquids, making the device highly user-friendly.

Moreover, the independent design of the medicinal liquid chamber and the ultrasonic therapeutic unit ensures that the two components do not interfere with each other, significantly improving the hygiene and lifespan of the device. The high-frequency ultrasonic vibrations produced by the therapeutic unit not only expel the liquid from the infiltrating medicinal liquid cotton pad through the outlet holes onto the skin but also stimulate the skin cells to accelerate movement. This facilitates the opening of skin pores and enhances the absorption of the medicinal liquid for targeted therapy.

The high-frequency vibrations also promote capillary movement in the treated area, break down blood clots through cavitation within the bloodstream, accelerate blood flow in capillaries, and stimulate metabolism. As a result, the invention achieves therapeutic, rehabilitative, and health-preserving effects on the applied body parts.

### BRIEF DESCRIPTION OF THE DRAWINGS

FIG. 1 is a structural schematic of the invention from the surface perspective;

FIG. 2 is a structural schematic of FIG. 1 showing the cover open and the infiltrating medicinal liquid cotton pad removed;

FIG. 3 is a structural schematic of the invention from the bottom perspective;

FIG. 4 is an exploded structural view of FIG. 3;

FIG. 5 is a cross-sectional structural view along direction A-A of FIG. 3;

FIG. 6 is the first exploded structural view of the ultrasonic therapeutic unit;

FIG. 7 is the second exploded structural view of the ultrasonic therapeutic unit;

FIG. 8 is a structural schematic of the ultrasonic therapeutic unit with a wireless charger;

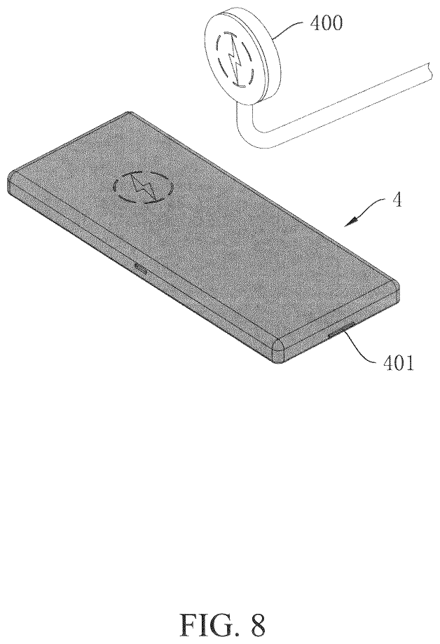

FIG. 9 is a structural schematic of the invention from FIG. 1 with an added strap.

### DETAILED DESCRIPTION OF THE INVENTION

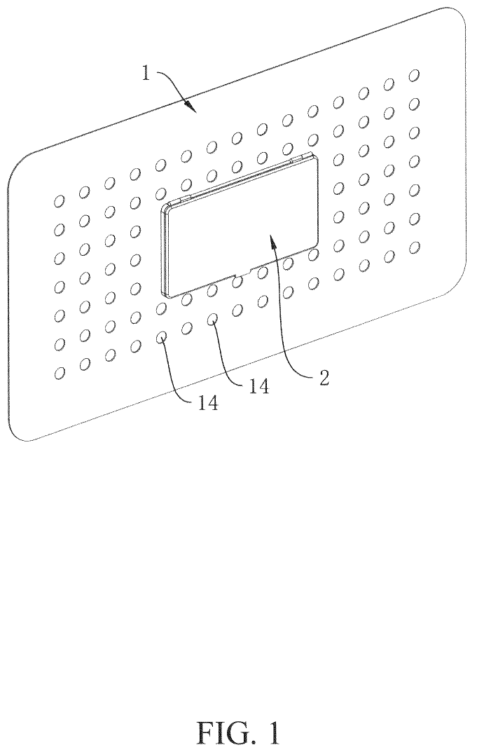

As shown in FIGS. 1 to 3, the described infiltrating medicinal liquid supply ultrasonic cavitation therapeutic patch device comprises a patch (1) and a medicinal application box (2) mounted on the patch. The medicinal application box (2) contains an infiltrating medicinal liquid cotton pad (3) and an ultrasonic therapeutic unit (4). The medicinal application box (2) includes a box body (21) and a box cover (22) that interlock with each other. The box body (21) further features a medicinal chamber (23) designed to house the infiltrating medicinal liquid cotton pad (3). The bottom surface of the box body (21) is equipped with several medicinal liquid outlet holes (24) that connect to the medicinal chamber (23). The box cover (22) is designed to open and close over the medicinal chamber (23), forming a built-in liquid supply mechanism (20) comprising the box cover (22), the infiltrating medicinal liquid cotton pad (3), the medicinal chamber (23), and the medicinal liquid outlet holes (24). The ultrasonic therapeutic unit (4) is mounted on the box body (21). The patch (1) is also equipped with several through-holes (11) corresponding to the medicinal liquid outlet holes (24).

In practice, the bottom surface of the patch (1) can be coated with an adhesive layer made of materials similar to those used for adhesive bandages. During use, the device can be affixed to the desired body part to perform therapy. When the ultrasonic therapeutic unit (4) is activated, its high-frequency vibrations resonate through the box body (21), causing the medicinal liquid in the cotton pad (3) to flow out through the medicinal liquid outlet holes (24) onto the skin. These vibrations facilitate the diffusion and absorption of the medicinal liquid into the skin.

In a specific embodiment, the medicinal application box (2) can be integrated with the patch (1) as a disposable unit. In this configuration, the medicinal application box (2) cannot be detached from the patch (1) and is intended for single use. After use, the entire unit can be discarded. The ultrasonic therapeutic unit (4), however, is a reusable component designed to be detachable from the medicinal application box (2). This allows the ultrasonic therapeutic unit (4) to be paired with multiple disposable medicinal application boxes (2) and patches (1) for repeated therapeutic use.

Alternatively, the medicinal application box (2) and patch (1) can be connected using screws or other attachment mechanisms, creating a structure in which the patch (1) and infiltrating medicinal liquid cotton pad (3) are disposable components, while the medicinal application box (2) and ultrasonic therapeutic unit (4) are reusable. Users can simply replace the patch (1) and cotton pad (3) as needed to meet different therapeutic requirements or replenish medicinal liquid. This design simplifies operation.

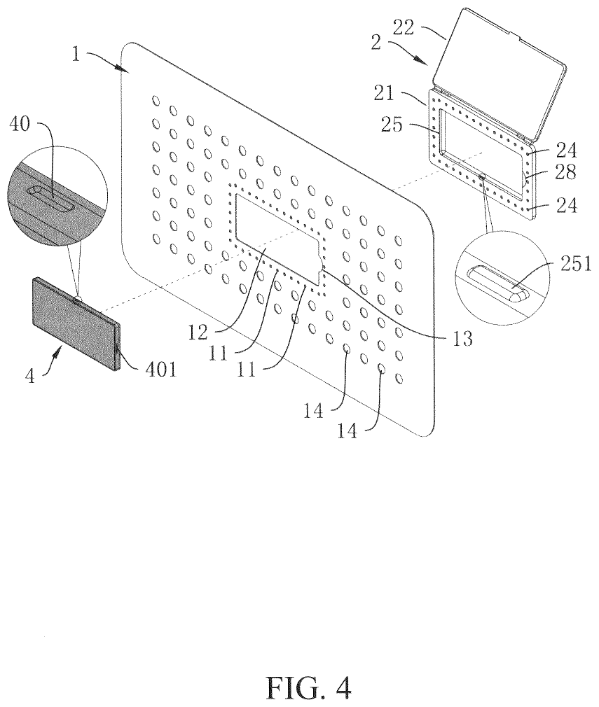

As shown in FIG. 4, the bottom surface of the box body (21) includes a cavity chamber (25) for embedding the ultrasonic therapeutic unit (4), which can be detachably mounted. Positioning the ultrasonic therapeutic unit (4) on the bottom of the box body (21) ensures direct contact with the skin during use, enabling efficient energy transfer for enhanced cavitation therapy.

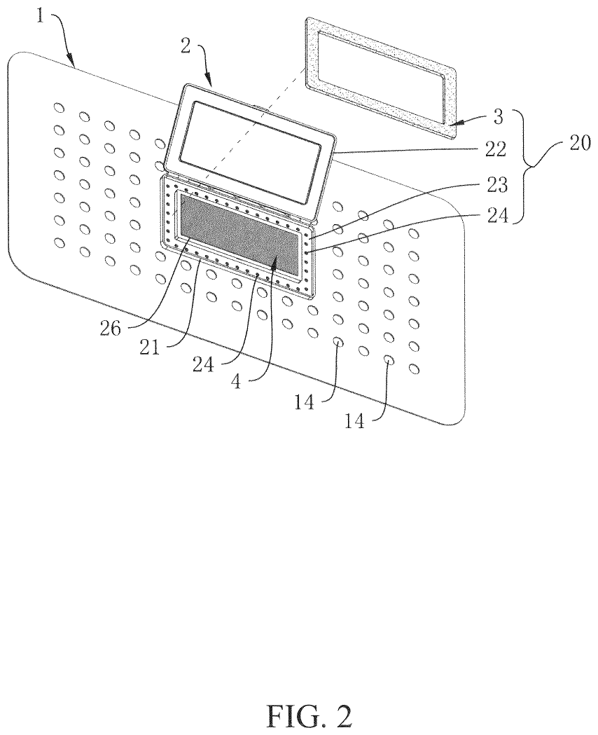

To ensure that the medicinal liquid cotton pad (3) surrounds the ultrasonic therapeutic unit (4) for uniform liquid application, the medicinal chamber (23) is designed as a ring around the cavity chamber (25), as shown in FIG. 2. The cotton pad (3) is correspondingly configured in a ring shape. This design evenly distributes the medicinal liquid on the skin surface and ensures better adhesion between the therapeutic unit (4) and the skin, improving energy transfer and therapeutic effects.

As shown in FIG. 4, snap-fit structures, including locking protrusions (251) and slots (40), are provided between the ultrasonic therapeutic unit (4) and the cavity chamber (25). Specifically, the slots (40) are located on the ultrasonic therapeutic unit (4), while the locking protrusions (251) are on the cavity chamber (25). These positions can be interchanged. The snap-fit design securely fastens the ultrasonic therapeutic unit (4) within the cavity chamber (25), preventing accidental detachment.

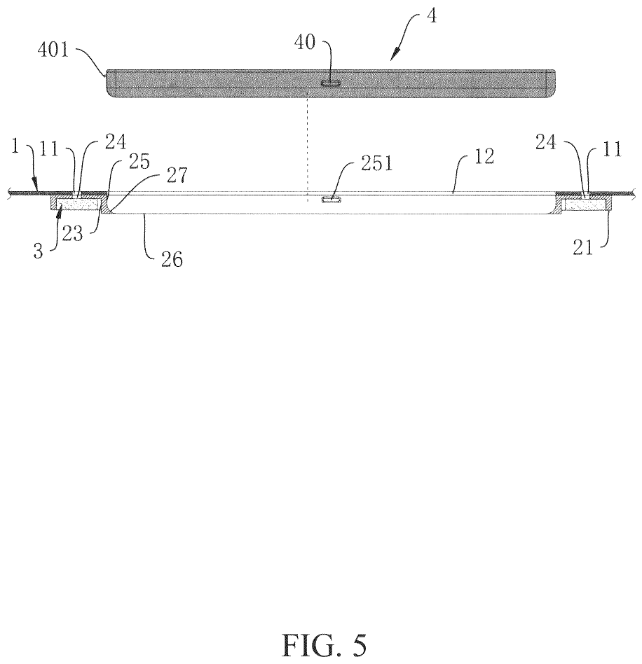

As shown in FIG. 5, the box body (21) features a through-hole (26) that connects to the cavity chamber (25) and includes a limiting step (27) between the two. The through-hole (26) reduces the weight and material usage of the box body (21), promoting environmental sustainability. Additionally, the through-hole (26) facilitates the removal of the ultrasonic therapeutic unit (4) from the cavity chamber (25) by allowing it to be pushed out more easily. The limiting step (27), in conjunction with the locking protrusions (251) and slots (40), stabilizes the therapeutic unit (4) within the cavity chamber (25), preventing unintended movement or detachment.

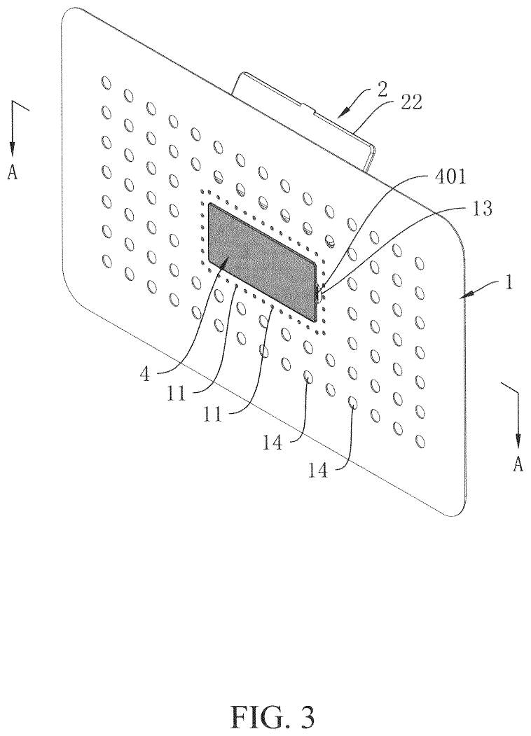

As shown in FIGS. 3 and 4, the patch (1) also includes perforations (12) corresponding to the cavity chamber (25), ensuring that the patch does not obstruct the insertion of the ultrasonic therapeutic unit (4) into the cavity chamber (25).

To facilitate the removal of the ultrasonic therapeutic unit (4), the bottom surface of the box body (21) includes a finger groove (28), and the patch (1) has a corresponding notch (13). The therapeutic unit (4) features a gripping protrusion (401). This design allows the ultrasonic therapeutic unit (4) to be easily removed by gripping the protrusion (401) through the groove (28) and pulling outward.

To enhance the breathability of the patch (1) and prevent discomfort, as shown in FIG. 1, the patch (1) is equipped with evenly spaced ventilation holes (14), which improve airflow and user comfort during use.

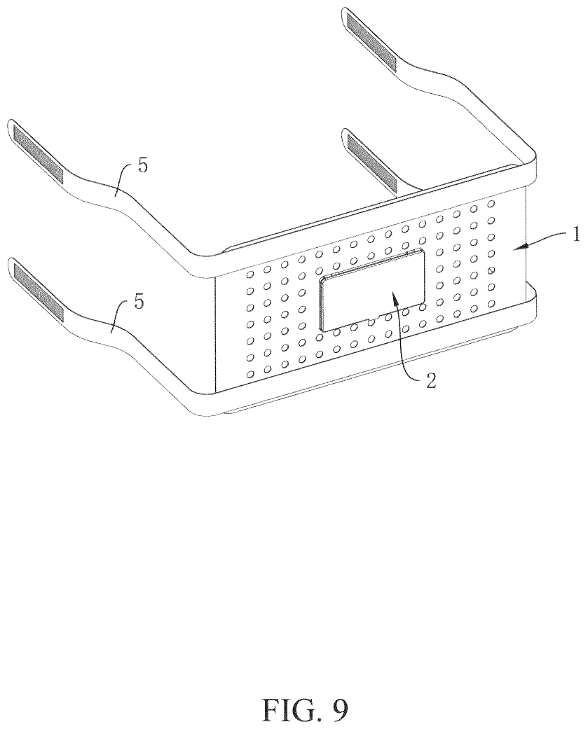

As shown in FIG. 9, the patch (1) may also feature straps (5). These straps (5) allow the device to be secured to body parts that are not suitable for adhesive application or where adhesion is unreliable. Both adhesive layers and straps (5) can be incorporated, or they can be used separately. Typically, two straps (5) are provided, as shown in FIG. 9, with one on each side of the patch (1). The straps (5) include hook-and-loop fasteners for convenient attachment.

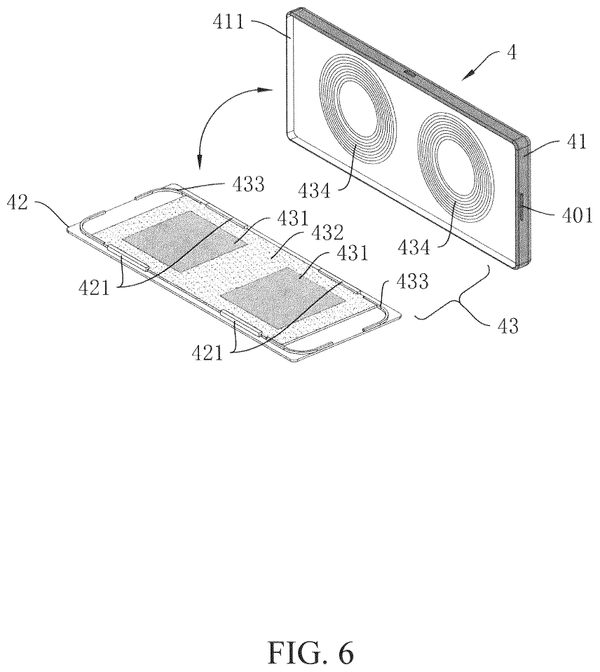

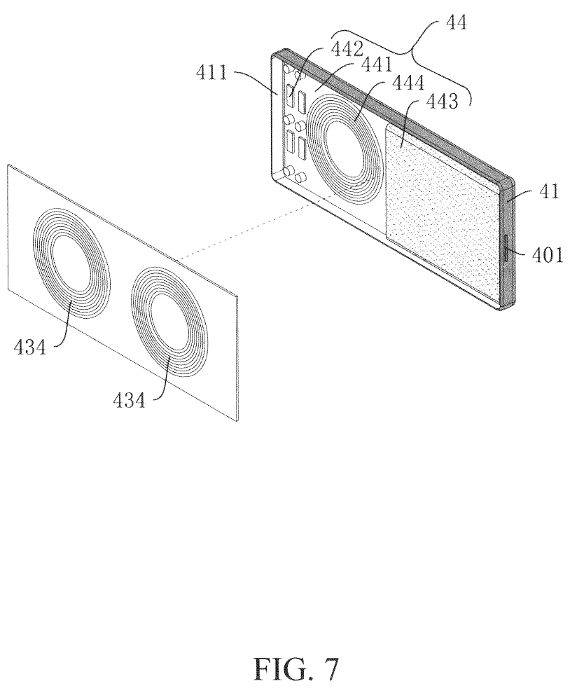

To improve the structure of the ultrasonic therapeutic unit (4), as shown in FIGS. 6 and 7, the therapeutic unit (4) includes a bottom shell (41) and a shell cover (42) with an accommodating cavity (411). These components house an ultrasonic vibration assembly (43) and a driving control assembly (44). The vibration assembly (43) comprises a sliding block (432) with a permanent magnet (431), elastic pieces (433) for buffering and rebound, and a driving coil (434) to generate magnetic motion. The driving control assembly (44) includes a circuit board (441) with a wireless communication module (442), a battery (443), and a wireless charging coil (444). The driving coil (434) is electrically connected to the circuit board (441) to drive the vibration assembly (43). Elastic pieces (433) provide reverse force for the sliding block (432) during movement, enabling reciprocation. Wireless charging coil (444) allows convenient charging, and the wireless communication module (442) supports device control via Bluetooth or Wi-Fi.

In practical applications, the infiltrating medicinal liquid cotton pad (3) can absorb traditional Chinese medicinal liquid or Western medicinal liquid for therapeutic use.

I claim:

1. An infiltrating medicinal liquid supply ultrasonic cavitation therapeutic patch device, comprising:
a patch (1) and a medicinal application box (2) mounted on the patch (1), the medicinal application box (2) containing an infiltrating medicinal liquid cotton pad (3) and an ultrasonic therapeutic unit (4);
wherein the medicinal application box (2) comprises a box body (21) and a box cover (22) that interlock with each other, the box body (21) further including a medicinal chamber (23) configured to house the infiltrating medicinal liquid cotton pad (3), and a plurality of medicinal liquid outlet holes (24) on a bottom surface of the box body (21) that connect to the medicinal chamber (23);
wherein the box cover (22) is designed to open and close over the medicinal chamber (23), forming a built-in liquid supply mechanism (20) with the box cover (22), the infiltrating medicinal liquid cotton pad (3), the medicinal chamber (23), and the medicinal liquid outlet holes (24); and
wherein the ultrasonic therapeutic unit (4) is disposed on the box body (21).

2. The infiltrating medicinal liquid supply ultrasonic cavitation therapeutic patch device according to claim 1, wherein the bottom surface of the box body (21) further comprises a cavity chamber (25) configured to embed the ultrasonic therapeutic unit (4), and the ultrasonic therapeutic unit (4) is detachably mounted within the cavity chamber (25).

3. The infiltrating medicinal liquid supply ultrasonic cavitation therapeutic patch device according to claim 2, wherein the medicinal chamber (23) is arranged in a ring surrounding the cavity chamber (25).

4. The infiltrating medicinal liquid supply ultrasonic cavitation therapeutic patch device according to claim 2, wherein the ultrasonic therapeutic unit (4) and the cavity chamber (25) include snap-fit structures comprising locking protrusions (251) and slots (40) that engage with each other.

5. The infiltrating medicinal liquid supply ultrasonic cavitation therapeutic patch device according to claim 2, wherein the box body (21) further comprises a through-hole (26) that connects to the cavity chamber (25), and a limiting step (27) formed between the through-hole (26) and the cavity chamber (25).

6. The infiltrating medicinal liquid supply ultrasonic cavitation therapeutic patch device according to claim 1, wherein the patch (1) further comprises perforations (12) corresponding to the cavity chamber (25).

7. The infiltrating medicinal liquid supply ultrasonic cavitation therapeutic patch device according to claim 6, wherein the bottom surface of the box body (21) further comprises a finger groove (28) connected to the cavity chamber (25);
wherein the patch (1) comprises a notch (13) corresponding to the finger groove (28); and
wherein the ultrasonic therapeutic unit (4) comprises a gripping protrusion (401).

8. The infiltrating medicinal liquid supply ultrasonic cavitation therapeutic patch device according to claim 1, wherein the patch (1) comprises a plurality of evenly spaced ventilation holes (14).

9. The infiltrating medicinal liquid supply ultrasonic cavitation therapeutic patch device according to claim 1, wherein the patch (1) further comprises straps (5).

10. The infiltrating medicinal liquid supply ultrasonic cavitation therapeutic patch device according to claim 1, wherein the ultrasonic therapeutic unit (4) comprises:
a bottom shell (41) and a shell cover (42) that interlock with each other and form an accommodating cavity (411);
an ultrasonic vibration assembly (43) and a driving control assembly (44) arranged within the accommodating cavity (411);
wherein the ultrasonic vibration assembly (43) comprises a sliding block (432) with a permanent magnet (431), elastic pieces (433) at both ends of the sliding block (432) for buffering and rebound, and a driving coil (434) configured to generate magnetic motion in cooperation with the permanent magnet (431);
wherein the driving control assembly (44) comprises a circuit board (441) with a wireless communication module (442), a battery (443) electrically connected to the circuit board (441), and a wireless charging coil (444);
and wherein the driving coil (434) is electrically connected to the circuit board (441) to drive the ultrasonic vibration assembly (43).
